(19) United States
(12) Patent Application Publication
(10) Pub. No.: US 2026/0026751 A1
(43) Pub. Date: Jan. 29, 2026

(54) IMPLANTABLE MEDICAL DEVICE FOR CARDIAC MONITORING USING SOUND

(71) Applicant: Medtronic, Inc., Minneapolis, MN (US)

(72) Inventors: Catherine R. Condie, Shoreview, MN (US); Joshua J. Blauer, White Bear Township, MN (US); Steven G. Nelson, Minneapolis, MN (US); Nicholas H. Finstrom, Coon Rapids, MN (US); Maius H. Wong, Blaine, MN (US); Leonardo Rapallini, Edina, MN (US)

(21) Appl. No.: 19/277,564

(22) Filed: Jul. 23, 2025

Related U.S. Application Data
(60) Provisional application No. 63/675,990, filed on Jul. 26, 2024.

(51) Int. Cl. A61B 5/00; A61B 5/02

(52) U.S. Cl. CPC A61B 5/6869; A61B 5/0028; A61B 5/0031; A61B 5/02; A61B 5/7217; A61B 2562/0204; A61B 2562/0219

(57) ABSTRACT

An implantable medical device includes one or more sensors configured to capture at least one of a sound or a vibration emitted by a passive implanted device implanted within a patient. Processing circuitry may obtain, from the one or more sensors, a signal indicative of the at least one of the sound or the vibration captured by the one or more sensors, determine, based on the at least one of the sound or the vibration captured by the one or more sensors, a physiological characteristic of the patient, and generate an output based on the physiological characteristic of the patient.

### RELATED APPLICATIONS

This application claims the benefit of U.S. Provisional Application Ser. No. 63/675,990, filed Jul. 26, 2024, the entire contents of each of which are incorporated herein by reference.

### TECHNICAL FIELD

The disclosure relates generally to medical devices and, more particularly, medical device configured for heart monitoring.

### BACKGROUND

Some types of medical systems may monitor various patient data of a patient or a group of patients to detect changes in health. In some examples, the medical system may monitor the data to detect one or more health conditions, such as arrhythmia, heart failure, congestion, etc. In some examples, the medical system may include one or more of an implantable medical device or a wearable device to collect the data based on sensing of physiological or other parameters of the patient.

### SUMMARY

In general, aspects of this disclosure are directed to an implantable medical device (IMD) configured to detect sounds and/or vibrations to determine physiological characteristics of a patient. The implantable medical device may include an accelerometer and/or a microphone that can capture high frequency sounds and/or vibrations, such as sounds and/or vibrations over 100 Hertz or over 1000 Hertz.

In some examples, the implantable medical device is used in conjunction with a passive implanted device implanted within the patient. The passive implanted device is designed to emit sounds and/or vibrations that indicate the rate of blood flow and/or the type of blood flow through or around the passive implanted device. The implantable medical device can capture sounds and/or vibrations emitted by such a passive implanted device and to determine, based on the sounds and/or vibrations emitted by the passive implanted device, physiological characteristics of the patient such as the rate of blood flow and/or the type of blood flow at the site the passive implanted device within the heart of the patient.

In some aspects, the techniques described herein relate to an implantable medical device including: one or more sensors configured to capture at least one of a sound or a vibration emitted by a passive implanted device implanted within a patient; and processing circuitry configured to: obtain, from the one or more sensors, a signal indicative of the at least one of the sound or the vibration captured by the one or more sensors; determine, based on the at least one of the sound or the vibration captured by the one or more sensors, a physiological characteristic of the patient; and generate an output based on the physiological characteristic of the patient.

In some aspects, the techniques described herein relate to a method including: capturing, by one or more sensors of an implantable medical device, at least one of a sound or a vibration emitted by a passive implanted device implanted within a patient; obtaining, by processing circuitry and from the one or more sensors, a signal indicative of the at least one of the sound or the vibration captured by the one or more sensors; determining, by the processing circuitry and based on the at least one of the sound or the vibration captured by the one or more sensors, a physiological characteristic of the patient; and generating, by the processing circuitry, an output based on the physiological characteristic of the patient.

In some aspects, the techniques described herein relate to a system including: an implantable medical device including one or more sensors configured to capture at least one of a sound or a vibration emitted by a passive implanted device implanted within a patient; and an external device including processing circuitry configured to: obtain, from the one or more sensors, a signal indicative of the at least one of the sound or the vibration captured by the one or more sensors; determine, based on the at least one of the sound or the vibration captured by the one or more sensors, a physiological characteristic of the patient; and generate an output based on the physiological characteristic of the patient.

In some aspects, the techniques described herein relate to an apparatus including: means for capturing at least one of a sound or a vibration emitted by a passive implanted device implanted within a patient; means for obtaining a signal indicative of the at least one of the sound or the vibration captured by the one or more sensors; means for determining, based on the at least one of the sound or the vibration captured by the one or more sensors, a physiological characteristic of the patient; and means for generating an output based on the physiological characteristic of the patient.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with techniques of this disclosure.

FIG. 2A is a perspective drawing illustrating an example insertable cardiac monitor, in accordance with techniques of this disclosure.

FIG. 2B is a perspective drawing illustrating another example insertable cardiac monitor, in accordance with techniques of this disclosure.

FIG. 3 is a functional block diagram illustrating an example configuration of an example medical device, in accordance with techniques of this disclosure.

FIG. 4 is a functional block diagram illustrating an example configuration of an example external device, in accordance with techniques of this disclosure.

FIG. 5 is a functional block diagram illustrating an example configuration of a health monitoring system, in accordance with techniques of this disclosure.

FIG. 6 is a block diagram illustrating an example system that includes a network and computing devices, in accordance with techniques of this disclosure.

FIG. 7 is a flow diagram illustrating an example technique of determining a physiological characteristic of the patent.

FIG. 8 is a flow diagram that illustrates performing an example technique according to this disclosure.

### DETAILED DESCRIPTION

In general, aspects of this disclosure are directed to an implantable medical device (IMD) configured to determine a physiological characteristic of a patient based on sounds and/or vibrations detected by the implantable medical device. The implantable medical device may include sensors, such as a motion sensor and/or a microphone that can capture high frequency sounds and/or vibrations, such as sounds and/or vibrations over hundreds and/or thousands of hertz, or even sounds beyond the range of human perception, which may typically be between twenty hertz and twenty kilohertz.

Example implantable medical devices in accordance with techniques of this disclosure may include an insertable cardiac monitor such as the Reveal LINQ™ or LINQ II™ Insertable Cardiac Monitor (ICM), available from Medtronic, Inc. of Minneapolis, MN, a pacemaker/defibrillator, and the like. In some examples, the processing circuitry of a system including the medical device may determine one or more physiological characteristics of a patient based on capturing high frequency sounds and/or vibrations emitted by the heart of the patient. The implantable medical device may process the captured high frequency sounds and/or vibrations to detect heart conditions such as aortic stenosis or aortic regurgitation and/or to predict, based on the sounds and/or vibrations, heart failure, aneurism, and/or when Abdominal Aortic Aneurysm (AAA) grafts or transcatheter aortic valve replacement (TAVR) may be needed for the patient.

In some examples, an implantable medical device may be paired with a passive implanted device, such as a stent, a prosthetic heart valve, a graft, or an atrial appendage occlusion device that is implanted in or near the heart of a patient. The passive implanted device may emit signals, such as sounds or vibrations, indicative of motion, such as physical deformation of the implanted medical device. For example, the passive implanted device may be designed to alter the type and/or characteristics of signals emitted by the passive implanted device based on the flow rate of blood through or around the passive implanted device and/or based on the flow type of blood through or around passive implanted device. The implantable medical device may capture sounds and/or vibrations emitted by the passive implanted device to determine physiological characteristics of the patient, such as the flow rate and/or flow type of blood through or around the passive implanted device.

By capturing high frequency sounds and vibrations, the techniques described herein may enable implantable medical devices to capture subtle physiological changes in the hearts of patients, which may enable early detection of cardiac anomalies. Further, capturing high frequency sounds may enable implantable medical devices to better distinguish between different types of cardiac events and conditions. Being able to better distinguish between different types of cardiac events and conditions may enable more accurate diagnosis of cardiac conditions in the patients.

Further, by using an implantable medical device to capture high frequency sounds and vibrations emitted within patients, the techniques described in this disclosure may provide a minimally invasive solution for monitoring the cardiac health of patients compared to existing techniques such as Holter monitors or echocardiograms. Such existing techniques may be cumbersome and less comfortable for patients compared to the techniques described in this disclosure.

In addition, a passive implanted device that emits sounds or vibrations indicative of the flow rate of blood through or around the passive implanted device and/or the flow type of blood through or around passive implanted device may enable an implantable medical device that is able to capture high frequency sounds to detect abnormalities in the rate and type of blood flow in the hearts of patients. In particular, the sounds and vibrations emitted by a passive implanted device may enable the implantable medical device to perform localized monitoring of the hearts of patients to ensure that areas around the passive implanted device are closely observed for any complications caused by the passive implanted device.

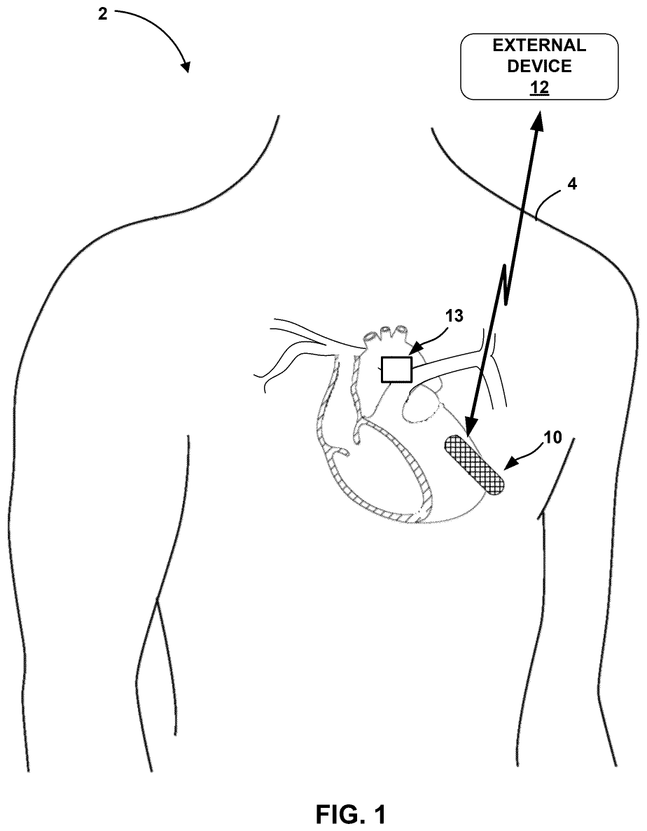

FIG. 1 illustrates the environment of an example medical system 2 in conjunction with a patient 4, in accordance with one or more techniques of this disclosure. As shown in FIG. 1, medical system 2 includes an implantable medical device (IMD) 10 that may be in wireless communication with external device 12. In some examples, medical system 2 and/or external device 12 may be in wired or wireless communication with other devices not pictured in FIG. 1.

External device 12 may be a computing device with a display viewable by the user and an interface for receiving user input to external device 12. In some examples, external device 12 may be a notebook computer, tablet computer, workstation, one or more servers, cellular phone, personal digital assistant, or another computing device that may run an application that enables the computing device to interact with IMD 10. External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

External device 12 may be used to configure operational parameters and/or device settings for IMD 10. External device 12 may be used to retrieve data from IMD 10. The retrieved data may include values of physiological parameters measured by IMD 10, indications of health conditions detected by IMD 10, and physiological signals recorded by IMD 10. As will be discussed in greater detail below, one or more remote computing devices may interact with IMD 10 in a manner similar to external device 12, e.g., to program IMD 10 and/or retrieve data from IMD 10, via a network.

IMD 10 is a medical device implanted in patient 4. In some examples, IMD 10 is implanted in the heart of patient 4. In some examples, IMD 10 is implanted outside of a thoracic cavity of patient 4 (e.g., subcutaneously in the pectoral location illustrated in FIG. 1). In some examples, IMD 10 may be positioned near the sternum near or just below the level of the heart of patient 4, e.g., at least partially within the cardiac silhouette. In some examples, IMD 10 may be positioned on other locations, such as patient 4's cranium region. In some examples, IMD 10 takes the form of the Reveal LINQ™ or LINQ II™ ICM. In some examples, the one or more sensors are configured to sense patient activity, e.g., one or more accelerometers.

IMD 10 includes one or more sensors (not shown in FIG. 1) and is configured to sense data via the one or more sensors. For example, IMD 10 may be configured to capture sounds, vibrations, and or other signals (e.g., electrical signals) emitted by the heart of patient 4 and/or by another medical device implanted in patient 4. To capture such sounds, vibrations, and/or other signals, IMD 10 may include one or more sensors, such as an accelerometer and/or a microphone.

The one or more sensors may be configured to continuously (e.g., in a periodic and/or event-driven manner) sense for and capture signals, such as signals emitted by the heart of patient 4 and/or by another medical device implanted in patient 4. The one or more sensors of IMD 10 may be capable of capturing higher frequency signals, which may be sounds and/or vibrations that are higher than one hundred hertz or are higher than one thousand hertz.

IMD 10 is configured to process the signals captured by the one or more sensors to determine, based on the captured signals, one or more physiological characteristics of the heart of patient 4. IMD 10 may generate an output based on the one or more physiological characteristics of the heart of patient 4. For example, IMD 10 may generate an output signal indicative of the one or more physiological characteristics of the heart of patient 4 and may output the signal to external device 12.

IMD 10 may be configured to capture heart sounds emitted by the heart of patient 4 and to determine, based on the higher frequency sounds emitted by the heart of patient 4 and captured by the one or more sensors, information regarding the cardiac health of patient 4. For example, IMD 10 may be configured to detect, based on the higher frequency heart sounds captured by IMD 10, heart conditions such as aortic stenosis or aortic regurgitation and/or to predict, based on the higher frequency signals, heart failure or aneurism. In some examples, IMD 10 may be configured to predict, based on the captured higher frequency heart sounds, when Abdominal Aortic Aneurysm (AAA) grafts or transcatheter aortic valve replacement (TAVR) may be needed for patient 4.

As described above, IMD 10 may be configured to capture sounds, vibrations, and/or other signals emitted by another medical device implanted in patient 12. In the example of FIG. 1, implanted medical device 13 may be implanted in patient 4, such as in the heart of patient 4 or elsewhere within patient 4.

In some examples, implanted medical device 13 is a passive implanted device, which is a device that may function without an external power source or active electronics. That is, a passive implanted device may not require electrical power to operate and may instead rely on their material properties and design to perform their intended functions. Examples of a passive implanted device may include a stent, a prosthetic heart valve, a graft, or an atrial appendage occlusion device.

Implanted medical device 13 is configured to emit sounds and/or vibrations that can be captured by the one or more sensors of IMD 10. For example, implanted medical device 13 may be configured to output sounds or vibrations indicative of motion, such as deformation of implanted medical device 13. For example, implanted medical device 13 may output sounds or vibrations indicative of whether implanted medical device 13 is deforming or not deforming as anticipated.

In some examples, implanted medical device 13 may include an audio output device, such as a speaker device that may emit sounds indicative of motion. Implanted medical device 13 may include a piezoelectric element that is able to convert mechanical motion into electrical energy. The piezoelectric element may be powered by the motion of implanted medical device 13 and may therefore be capable of powering a circuit element (e.g., a speaker device or a transmitter device) to emit a signal (e.g., an audible and/or electric signal).

In some examples, implanted medical device 13 may be configured or otherwise designed to alter the type and/or characteristics of signals emitted by implanted medical device 13, such as the spectrum emission of sounds or other signals, based on the physiological characteristic of patient 4. In some examples, implanted medical device 13 may be configured of designed (e.g., via the physical design of implanted medical device 13) to alter the type and/or characteristics of signals emitted by implanted medical device 13 based on the flow rate of blood through or around implanted medical device 13 and/or based on the flow type (e.g., laminar flow versus turbulent flow) of blood through or around implanted medical device 13. In this way, implanted medical device 13 may be able to output signals indicative of the flow rate and/or flow type of blood through or around implanted medical device 13.

Further, by altering the type and/or characteristics of signals emitted by implanted medical device 13 based on the flow rate and/or flow type of blood through or around implanted medical device 13, implanted medical device 13 may be able to emit signals indicative of various physiological characteristics of patient 4 such as calcification, thrombus formation, occlusion, and the like. The formation of calcium, the development of blood clots, and the blockage or closing of a blood vessel may each impact blood flow characteristics, such as the flow rate and/or flow type, for patient 4.

In some examples, implantable medical device 13, such as a graft, may include an inert, biocompatible, dissolvable substrate, such as nitrogen bubbles. Implantable medical device 13 may be configured to, while implanted in patient 4, release the nitrogen bubbles. The release of such bubbles may amplify the differences between the types of flows. In this way, the release of nitrogen bubbles may improve the ability IMD 10 to discriminate the sounds emitted by implantable medical device 13.

In some examples, components of medical system 2, such as IMD 10 and/or external device 12, may use the thermal properties of implantable medical device 13 to evaluate blood flow around implantable medical device 13. Component of medical system 2 may be able to evaluate blood flow around implantable medical device 13 based on implantable medical device 13's thermal properties due to differences in thermal properties between turbulent flow and laminar flow and differences in thermal properties for different velocities of laminar flow.

Components of medical system 2 may be used to externally heat implantable medical device 13. For example, medical system 2 may include a device configured to externally heat implantable medical device 13 via ultrasound, or may include a heat delivery system such as a catheter configured to heat implantable medical device 13.

After implantable medical device 13 is heated, IMD 10 and/or external device 12 may be configured to monitor the rate of cooling of implantable medical device 13 to determine if the blood flow around implantable medical device 13 is a laminar flow, the flow velocity of the blood flow, and/or whether the blood flow is turbulent or occluded. IMD 10 and/or external device 12 may be configured to monitor the temperature of implantable medical device 13 using a thermistor, a thermocouple on implantable medical device 13, a non-contact thermal sensing device such as infrared or forward looking infrared (FLIR), or a device that detects impedance changes of implanted medical device 13 (e. g., measured via IMD 10 or via external device 12) as the temperature of a material changes. This may be used to help place implantable medical device 13 in an acute setting and to monitor the implantable medical device 13 during transcatheter placement.

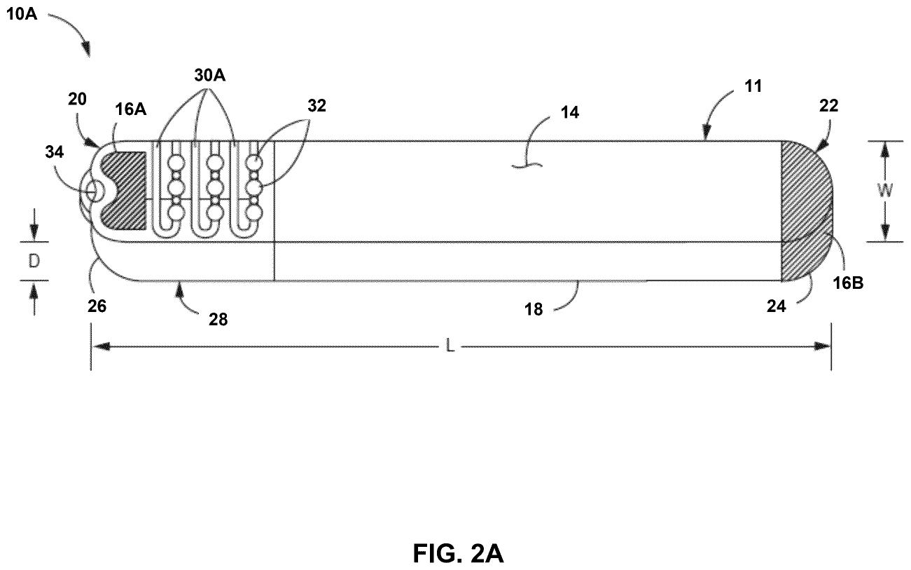

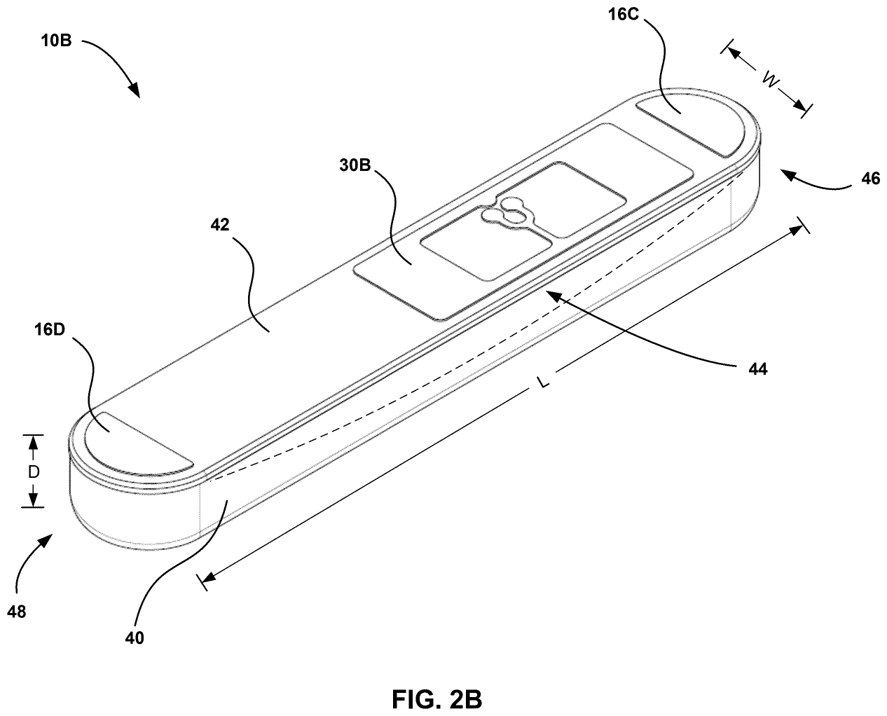

FIG. 2A is a perspective drawing illustrating an IMD 10A, which may be an example configuration of IMD 10 of FIG. 1 as an ICM. In the example shown in FIG. 2A, IMD 10A may be embodied as a monitoring device having housing 11, proximal electrode 16A and distal electrode 16B. Housing 11 may further comprise first major surface 14, second major surface 18, proximal end 20, and distal end 22. Housing 11 encloses electronic circuitry located inside the IMD 10A and protects the circuitry contained therein from body fluids. Housing 11 may be hermetically sealed and configured for subcutaneous implantation. Electrical feedthroughs provide electrical connection of electrodes 16A and 16B.

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of patient 4 using a minimally invasive procedure and to remain in the desired orientation during insertion. For example, the device shown in FIG. 2A includes radial asymmetries (notably, the rectangular shape) along the longitudinal axis that maintains the device in the proper orientation following insertion. For example, the spacing between proximal electrode 46A and distal electrode 46B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 14 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 2A, once inserted within patient 4, the first major surface 14 faces outward, toward the skin of patient 4 while the second major surface 18 is located opposite the first major surface 14. In addition, in the example shown in FIG. 2A, proximal end 20 and distal end 22 are rounded to reduce discomfort and irritation to surrounding tissue once inserted under the skin of patient 4. IMD 10A, including instrument and method for inserting IMD 10 is described, for example, in U.S. Patent Publication No. 2014/0276928, incorporated herein by reference in its entirety.

Proximal electrode 16A is at or proximate to proximal end 20, and distal electrode 16B is at or proximate to distal end 22. Proximal electrode 16A and distal electrode 16B are used to sense cardiac EGM signals, e.g., ECG signals, thoracically outside the ribcage, which may be sub-muscularly or subcutaneously. EGM signals may be stored in a memory of IMD 10A, and data may be transmitted via integrated antenna 30A to another device, which may be another implantable device or an external device, such as external device 12. In some example, electrodes 16A and 16B may additionally or alternatively be used for sensing any bio-potential signal of interest, which may be, for example, an EGM, EEG, EMG, or a nerve signal, or for measuring impedance, from any implanted location.

In the example shown in FIG. 2A, proximal electrode 16A is at or in close proximity to the proximal end 20 and distal electrode 16B is at or in close proximity to distal end 22. In this example, distal electrode 16B is not limited to a flattened, outward facing surface, but may extend from first major surface 14 around rounded edges 24 and/or end surface 26 and onto the second major surface 18 so that the electrode 16B has a three-dimensional curved configuration. In some examples, electrode 16B is an uninsulated portion of a metallic, e.g., titanium, part of housing 11.

In the example shown in FIG. 2A, proximal electrode 16A is located on first major surface 14 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 14 similar to that shown with respect to proximal electrode 16A.

The various electrode configurations allow for configurations in which proximal electrode 16A and distal electrode 16B are located on both first major surface 14 and second major surface 18. In other configurations, such as that shown in FIG. 2A, only one of proximal electrode 16A and distal electrode 16B is located on both major surfaces 14 and 18, and in still other configurations both proximal electrode 16A and distal electrode 16B are located on one of the first major surface 14 or the second major surface 18 (e.g., proximal electrode 16A located on first major surface 14 while distal electrode 16B is located on second major surface 18). In another example, IMD 10A may include electrodes on both major surface 14 and 18 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 2A, proximal end 20 includes a header assembly 28 that includes one or more of proximal electrode 16A, integrated antenna 30A, anti-migration projections 32, and/or suture hole 34. Integrated antenna 30A is located on the same major surface (i.e., first major surface 14) as proximal electrode 16A and is also included as part of header assembly 28. Integrated antenna 30A allows IMD 10A to transmit and/or receive data. In other examples, integrated antenna 30A may be formed on the opposite major surface as proximal electrode 16A, or may be incorporated within the housing 11 of IMD 10A. In the example shown in FIG. 2A, anti-migration projections 32 are located adjacent to integrated antenna 30A and protrude away from first major surface 14 to prevent longitudinal movement of the device. In the example shown in FIG. 2A, anti-migration projections 32 include a plurality (e.g., nine) small bumps or protrusions extending away from first major surface 14. As discussed above, in other examples anti-migration projections 32 may be located on the opposite major surface as proximal electrode 16A and/or integrated antenna 30A. In addition, in the example shown in FIG. 2A, header assembly 28 includes suture hole 34, which provides another means of securing IMD 10A to patient 4 to prevent movement following insertion. In the example shown, suture hole 34 is located adjacent to proximal electrode 16A. In one example, header assembly 28 is a molded header assembly made from a polymeric or plastic material, which may be integrated or separable from the main portion of IMD 10A.

FIG. 2B is a perspective drawing illustrating another IMD 10B, which may be another example configuration of IMD 10 from FIG. 1 as an ICM. IMD 10B of FIG. 2B may be configured substantially similarly to IMD 10A of FIG. 2A, with differences between them discussed herein.

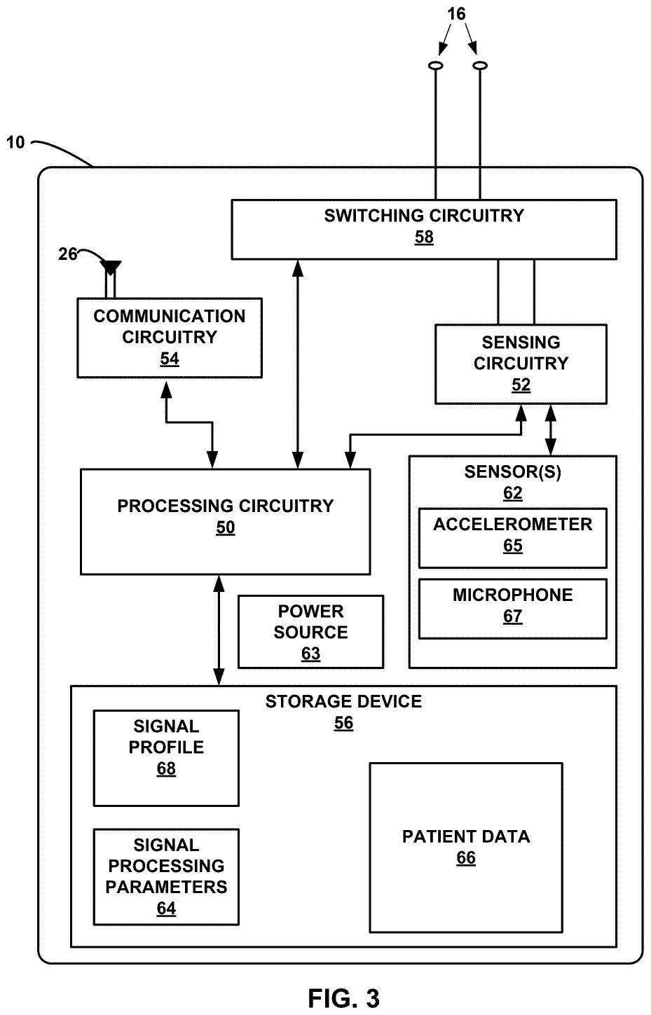

IMD 10B may include a leadless, subcutaneously-implantable monitoring device, e.g. an ICM. IMD 10B includes housing having a base 40 and an insulative cover 42. Proximal electrode 16C and distal electrode 16D may be formed or placed on an outer surface of cover 42. Various circuitries and components of IMD 10B, e.g., described below with respect to FIG. 3, may be formed or placed on an inner surface of cover 42, or within base 40. In some examples, a battery or other power source of IMD 10B may be included within base 40. In the illustrated example, antenna 30B is formed or placed on the outer surface of cover 42, but may be formed or placed on the inner surface in some examples. In some examples, insulative cover 42 may be positioned over an open base 40 such that base 40 and cover 42 enclose the circuitries and other components and protect them from fluids such as body fluids. The housing including base 40 and insulative cover 42 may be hermetically sealed and configured for subcutaneous implantation.

Circuitries and components may be formed on the inner side of insulative cover 42, such as by using flip-chip technology. Insulative cover 42 may be flipped onto a base 40. When flipped and placed onto base 40, the components of IMD 10B formed on the inner side of insulative cover 42 may be positioned in a gap 44 defined by base 40. Electrodes 16C and 16D and antenna 30B may be electrically connected to circuitry formed on the inner side of insulative cover 42 through one or more vias (not shown) formed through insulative cover 42. Insulative cover 42 may be formed of sapphire (i.e., corundum), glass, parylene, and/or any other suitable insulating material. Base 40 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between proximal electrode 46C and distal electrode 46D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within patient 4, outer surface of cover 42 faces outward, toward the skin of patient 4. In addition, as shown in FIG. 2B, proximal end 46 and distal end 48 are rounded to reduce discomfort and irritation to surrounding tissue once inserted under the skin of patient 4. In addition, edges of IMD 10B may be rounded.

FIG. 3 is a functional block diagram illustrating an example configuration of IMD 10 of FIG. 1 in accordance with one or more techniques described herein. In the illustrated example, IMD 10 includes electrodes 16 (e.g., corresponding to any of electrodes 16A-16D), antenna 26, processing circuitry 50, sensing circuitry 52, communication circuitry 54, storage device 56, switching circuitry 58, and sensors 62. Processing circuitry 50 may be operatively coupled to sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples. IMD 10 further comprises a power source 64 to provide operational power for processing circuitry 50, sensing circuitry 52, communication circuitry 54, storage device 56, switching circuitry 58, and sensors 62.

Processing circuitry 50 may be configured to implement functionality and/or execute instructions within IMD 10. For example, processing circuitry 50 may receive and execute instructions that provide the functionality described herein, such as in FIG. 1. Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be configured to sense cardiac activity of patient 4. In some examples, sensing circuitry 52 may be selectively coupled to electrodes 16 via switching circuitry 58, e.g., to sense electrical signals of the heart of patient 4. For example, sensing circuitry may select electrodes 16 and polarity, referred to as the sensing vector, used to sense cardiac activity data (e.g., electrocardiogram (ECG) data, electrogram (EGM) data, etc.) as controlled by processing circuitry 50. Electrodes 16 may be configured to sense a parameter indicative of heart failure, and processing circuitry 50 may be configured to determine a risk of heart failure further based on the parameter indicative of heart failure. For example, electrodes 16 may measure subcutaneous tissue or interstitial impedance values, respiratory rate, heart rate (e.g., day and/or night heart rate), QRS morphology, HRV (e.g., day and/or night HRV), etc.

In some examples, sensing circuitry 52 may include one or more filters and amplifiers for filtering and amplifying signals received from electrodes 16 and/or sensors 62. Sensing circuitry 52 and processing circuitry 50 may store patient data in storage device 56, e.g., digitized samples of electrical signals. Sensing circuitry 52 may also monitor signals from sensors 62 and may send signals captured by sensors 62 to processing circuitry 50 for processing. Sensing circuitry 52 may capture sensor signals from any one of sensors 62, e.g., to produce other patient data, in order to facilitate monitoring of patient activity and detecting changes in patient health.

Sensors 62 may include one or more multi-axial accelerometers 65 (“accelerometer 65”), one or more microphones 67 (“microphone 67”), pressure sensors, and/or optical sensors, as examples. Accelerometer 65 and microphone 67 may each be configured to continuously (e.g., in a periodic and/or event-driven manner) sense for and capture signals, such sounds and/or vibrations emitted by the heart of patient 4 and sounds and/or vibrations emitted by implanted medical device 13.

Microphone 67 may be any device configured to capture sound and to convert the captured sound into an electrical signal that can be recorded, amplified, or otherwise processed. In some examples, microphone 67 may include a dynamic microphone, a piezoelectric microphone, and the like.

Accelerometer 65 may be any device configured to measure the proper acceleration of IMD 10. Accelerometer 65 may detect and capture vibrations, which may be in the form of small, rapid accelerations, and may convert the vibrations into an electrical signal. Because sound waves may cause vibrations that can be captured by accelerometer 65, accelerometer 65 may be able to capture sound. As such, in some examples, IMD 10 may use accelerometer 65 as microphone 67 or may use accelerometer 65 in conjunction with a dedicated microphone 67 to capture sound.

Sensors 62 may be configured to capture high frequency sounds, vibrations, or other signals emitted by the heart of patient 12 and/or implanted medical device 13. For example, such high frequency sounds, vibrations, or other signals may be signals having frequencies in the hundreds of hertz, in the thousands of hertz, or more. Sensors 62 may be designed to capture such high frequency signals.

Communication circuitry 54, which may be an example of the communication circuitry described in FIG. 1, may include any suitable hardware, firmware, software or any combination thereof for wirelessly communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, processing circuitry 50 may control communication circuitry 54 to transmit data to another device, e.g., external device 12 or a cloud computing system comprising one or more computing devices, for analysis, including the determining of various sleep properties of patient 4. In this manner, the techniques of this disclosure may advantageously enable improved accuracy in the detection of changes in patient health and, consequently, better evaluation of the condition of patient 4.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), dynamic random-access memory (DRAM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include patient data (e.g., physiological characteristics, sounds and vibrations captured by sensors 62, etc.).

Processing circuitry 50 may obtain signals from accelerometer 65 and/or microphone 67, such as by receiving, from sensors 62 and/or from sensing circuitry 52, signals captured by accelerometer 65 and/or microphone 67. As described above, accelerometer 65 and microphone 67 may continuously capture sounds and/or vibrations. Accelerometer 65 and microphone 67 may generate signals that are indicative of the captured sounds and/or vibrations, which processing circuitry 50 may obtain from sensors 62 and/or from sensing circuitry 52.

The signals generated by accelerometer 65 and microphone 67 indicative of the captured sounds and/or vibrations may encode or otherwise include the frequencies and amplitudes of the captured sounds and/or vibrations. Processing circuitry 50 may obtain and process the signals generated by accelerometer 65 and microphone 67 in order to determine physiological characteristics of patient 4 based on the signals.

In the example of signals generated by a multi-axis accelerometer, such as accelerometer 65, the signals may include, for each axis of accelerometer 65, a corresponding signal captured by the axis. Processing circuitry 50 may select the corresponding signals for one or more of the axes of accelerometer 65 for processing to determine a physiological characteristic of patient 4. For example, if accelerometer 65 outputs a corresponding signal for each of an x-axis, a y-axis, or a z-axis, processing circuitry 50 may select one or more of the three corresponding signals for the three axes of accelerometer 65 for processing to determine a physiological characteristic of patient 4.

In some examples, processing circuitry 50 may preform various digital signal processing techniques to filter the signals and to extract a specific frequency range of sounds and/or vibrations from the signals. For example, processing circuitry 50 may perform low-pass filtering to extract frequencies below a certain cutoff frequency, high-pass filtering to extract frequencies above a certain cutoff frequency, or band-pass filtering to extract a specific range of frequencies from the signals. Processing circuitry 50 may also sample the signals at a certain sampling rate and/or sensitivity.

Processing circuitry 50 may process signals obtained from sensors 62 to determine a physiological characteristic of patient 4. In some examples, processing circuitry 50 may determine, based on signals generated by accelerometer 65 and/or microphone 67 indicative of the sounds and/or vibrations emitted by the heart of patient, information regarding the cardiac health of patient 4. For example, processing circuitry 50 may, based on the signals generated by accelerometer 65 and/or microphone 67, detect one or more heart conditions of patient 4, such as aortic stenosis or aortic regurgitation, and/or predict, based on the signals, heart failure or aneurism. In some examples, processing circuitry 50 may, based on the signals generated by accelerometer 65 and/or microphone 67, predict when Abdominal Aortic Aneurysm (AAA) grafts or transcatheter aortic valve replacement (TAVR) may be needed for patient 4.

In some examples, processing circuitry 50 may determine, based on signals generated by accelerometer 65 and/or microphone 67 indicative of the sounds and/or vibrations emitted by another implanted medical device in patient 4, such as implanted medical device 13, a physiological characteristic of patient 4. For example, processing circuitry 50 may, based on the signals generated by accelerometer 65 and/or microphone 67, determine the motion of the implanted medical device, such as whether the implanted medical device is or is not deforming as anticipated. In another example, processing circuitry 50 may, based on the signals generated by accelerometer 65 and/or microphone 67, determine the rate of blood flow through and/or around the implanted medical device and/or the flow type (e.g., laminar flow or turbulent flow) of blood flow through and/or around the implanted medical device. In another example, processing circuitry 50 may, based on the signals generated by accelerometer 65 and/or microphone 67, determine various physiological characteristics of patient 4 such as calcification, thrombus formation, blood vessel occlusion, and the like.

Storage device 56 may store signal profile 68, which may be a data store that includes, for each of the physiological characteristics of patient 4 that is detectable based on the sounds and/or vibrations captured by sensors 62, information for processing the signals obtained from sensors 62 to detect the corresponding physiological characteristic. For example, signal profile 68 may specify, for each physiological characteristic to be detected, the source of the signal that is to be processed, such as microphone 67, accelerometer 65, or one or more specific axes of accelerometer 65. Signal profile 68 may also specify, for each physiological characteristic to be detected, a frequency range of the signal that is to be processed. For example, the information may specify a cut-off frequency below which the signal is attenuated, a cut-off frequency above which the signal is attenuated, or a particular frequency range outside of which the signal is attenuated.

Signal profile 68 may specify, for each physiological characteristic to be detected, a signature for the corresponding physiological characteristic, which may be a distinctive pattern or set of characteristics that processing circuitry 50 to identify the corresponding physiological characteristic based on sounds and/or vibrations captured by sensors 62. In some examples, a signature for a corresponding physiological characteristic may be a specific pattern of sounds and/or vibrations, which may include parameters such as frequency, amplitude, phase, duration, intensity, timing, rhythm, and the like. For example, the signature for a corresponding physiological characteristic may be specific sounds or patterns of sounds that can be recognized by processing circuitry 50 based on the parameters. Processing circuitry 50 may be able to perform such sound recognition or pattern matching based on changes in sound recorded over a frequency range and tracked for changes or patterns in the parameters.

Processing circuitry 50 may process signals obtained from sensors 62 based on the information stored in signal profile 68 to determine a physiological characteristic of patient 4. Processing circuitry 50 may receive, from sensors 62, a signal indicative of the sounds and/or vibrations captured by accelerometer 65 and/or microphone 67. Processing circuitry 50 may select one or more physiological characteristics of patient 4 to detect from the obtained signal. Processing circuitry 50 may, for each of the selected one or more physiological characteristics, process the signal according to the corresponding information for processing the signal, as specified by signal profile 68, to determine whether the signal includes the signature for the physiological characteristic. If processing circuitry 50 detects a signature for a physiological characteristic in the signal obtained from sensors 62, processing circuitry 50 may determine that the physiological characteristic is currently existing in patient 4.

In some examples, processing circuitry 50 may use one or more neural networks to detect, based on signals obtained from sensors 62, one or more physiological characteristics of patient 4. The one or more neural networks may include a deep neural network (DNN), a convolutional neural network (CNN), a recurrent neural network (RNN), a transformer-based network, and the like. The one or more neural networks may be trained using machine learning techniques, such as supervised learning, to receive a signal indicative of sounds and/or vibrations captured by accelerometers and/or microphones, such as accelerometer 65 and/or microphone 67, and to output one or more predicted physiological characteristics of a patient, such as a probability distribution of predicted physiological characteristics. Processing circuitry 50 may input a signal indicative of the sounds and/or vibrations captured by accelerometer 65 and/or microphone 67 into such one or more neural networks trained to predict physiological characteristics of a patient. The one or more neural networks may, in response, output or more predicted physiological characteristics of patient 4.

In some examples, processing circuitry 50 may compare a signal obtained from sensors 62 against a baseline signal to determine one or more physiological characteristics of patient 4. After IMD 10 is implanted in patient 4, accelerometer 65 and/or microphone 67 may capture ambient sounds and/or vibrations that are emitted within the body of patient 4. Processing circuitry 50 may determine, based on ambient sounds and/or vibrations that are emitted within the body of patient 4, a corresponding signature for the ambient sounds and/or vibrations that are emitted within the body of patient 4. As described above, a signature for the ambient sounds and/or vibrations may be a specific pattern of sounds and/or vibrations, which may include parameters such as frequency, amplitude, phase, duration, intensity, timing, rhythm, and the like.

After implanted medical device 13 is implanted in the body of patient 4, accelerometer 65 and/or microphone 67 may capture sounds and/or vibrations that are emitted within the body of patient 4. Such sounds and/or vibrations that are emitted within the body of patient 4 after implantation of implanted medical device 13 is referred to as a baseline signal for implanted medical device 13. Processing circuitry 50 may determine, based on sounds and/or vibrations that are emitted within the body of patient 4 after implantation of implanted medical device 13, a corresponding signature for the baseline signal for implanted medical device 13. As described above, a signature for the baseline signal for implanted medical device 13 may be a specific pattern of sounds and/or vibrations, which may include parameters such as frequency, amplitude, phase, duration, intensity, timing, rhythm, and the like over a particular period of time.

After determining the baseline signal for implanted medical device 13, accelerometer 65 and/or microphone 67 may continue to capture sounds and/or vibrations that are emitted within the body of patient 4, and sensors 62 may generate signals indicative of the sounds and/or vibrations captured by accelerometer 65 and/or microphone 67. Processing circuitry 50 may obtain the signals generated by sensors 62 indicative of the sounds and/or vibrations captured by accelerometer 65 and/or microphone 67 and may determine, based on the signature for the baseline signal for implanted medical device 13, one or more physiological characteristics of patient 4 indicated by the sounds and/or vibrations captured by accelerometer 65 and/or microphone 67.

For example, processing circuitry 50 may compare one or more characteristics of the sounds and/or vibrations captured by accelerometer 65 and/or microphone 67, such as a pattern of the captured sounds and/or vibrations, which may include parameters such as frequency, amplitude, phase, duration, intensity, timing, rhythm, and the like, against the signature for the baseline signal for implanted medical device 13 to determine one or more differences between the sounds and/or vibrations captured by accelerometer 65 and/or microphone 67 and the baseline signal. Such differences may be indicative of one or more physiological characteristics of patient 4, such as whether implanted medical device 13 is operating properly, the motion of implanted medical device 13, whether implanted medical device 13 is or is not deforming as anticipated the rate of blood flow through and/or around implanted medical device 13, the flow type (e.g., laminar flow or turbulent flow) of blood flow through and/or around implanted medical device 13, calcification of a blood vessel, thrombus formation in a blood vessel, blood vessel occlusion, and the like.

In some examples, processing circuitry 50 may use one or more neural networks to detect, based on comparing the one or more characteristics of the sounds and/or vibrations captured by accelerometer 65 and/or microphone 67 against the signature for the baseline signal for implanted medical device 13, one or more physiological characteristics of patient 4. The one or more neural networks may be trained using machine learning techniques, such as supervised learning, to receive the difference between a signature for a baseline signal for an implanted medical device and one or more characteristics of the sounds and/or vibrations and to output one or more predicted physiological characteristics of a patient, such as a probability distribution of predicted physiological characteristics. For example, the one or more neural networks may be trained to predict physiological characteristics of patients, such as whether an implanted medical device is operating properly, the motion of an implanted medical device, whether an implanted medical device is or is not deforming as anticipated the rate of blood flow through and/or around an implanted medical device, the flow type (e.g., laminar flow or turbulent flow) of blood flow through and/or around an implanted medical device, calcification of a blood vessel, thrombus formation in a blood vessel, blood vessel occlusion, and the like.

Processing circuitry 50 may determine the difference between the signature for the baseline signal for implanted medical device 13 and the one or more characteristics of the sounds and/or vibrations captured by accelerometer 65 and/or microphone 67 and may input the determined difference into the one or more neural networks trained to predict physiological characteristics of a patient. The one or more neural networks may, in response, output one or more predicted physiological characteristics of patient 4.

Processing circuitry 50 may output an indication of one or more physiological characteristics of patient 4, such as a signal indicative of one or more physiological characteristics of patient 4. In some examples, processing circuitry 50 may output, using communication circuitry 54, a signal indicative of one or more physiological characteristics of patient 4 to external device 12 or to a remote server (e.g., a health monitoring system). Processing circuitry 50 may output the signal to external device 12 or to a remote server via any suitable wireless communication scheme, such as NFC, RF communication, Bluetooth, WiFi, and the like. In some examples, processing circuitry 50 may also store the determined one or more physiological characteristics of patient 4 as patient data 66 in storage device 56.

In the example above, the techniques of this disclosure are described as being performed by processing circuitry 50. However, the techniques, at least in part, may be performed by other processing circuitry of system 2, such as processing circuitry of external device 12, processing circuitry of a remote server (e.g., a health monitoring system), and/or other processing circuitry. For example, processing circuitry of external device 12 and/or processing circuitry of a remote server may be configured to receive one or more signals generated by one or more sensors 62 and to determine, based on the one or more signals, one or more physiological characteristics of patient 4.

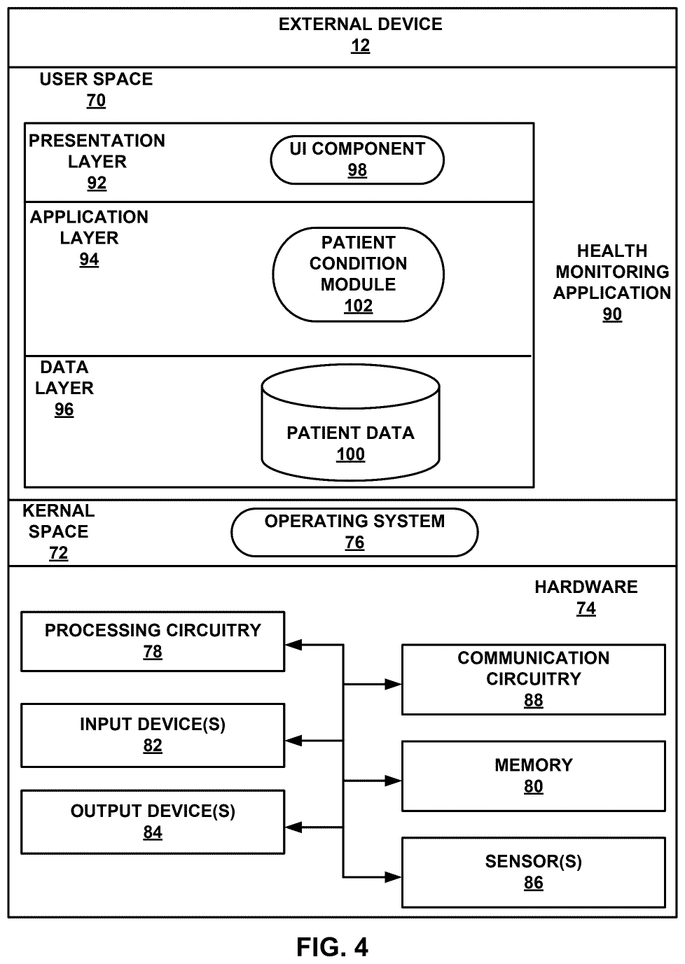

FIG. 4 is a block diagram illustrating an example configuration of external device 12, which, includes a smartphone, a laptop, a tablet computer, a personal digital assistant (PDA), a smartwatch, or any other suitable computing device. As shown in the example of FIG. 4, external device 12 may be logically divided into user space 70, kernel space 72, and hardware 74. Hardware 74 may include one or more hardware components that provide an operating environment for components executing in user space 70 and kernel space 72. User space 70 and kernel space 72 may represent different sections or segmentations of memory, where kernel space 72 provides higher privileges to processes and threads than user space 70. For instance, kernel space 72 may include operating system 76, which operates with higher privileges than components executing in user space 70.

As shown in FIG. 4, hardware 74 includes processing circuitry 78, memory 80, one or more input devices 82, one or more output devices 84, one or more sensors 86, and communication circuitry 88. Although shown in FIG. 4 as a stand-alone device for purposes of example, external device 12 may be any component or system that includes processing circuitry or other suitable computing environment for executing software instructions and, for example, need not necessarily include one or more elements shown in FIG. 4.

Processing circuitry 78 is configured to implement functionality and/or process instructions for execution within external device 12. For example, processing circuitry 78 may be configured to receive and process instructions stored in memory 80 that provide functionality of components included in kernel space 72 and user space 70 to perform one or more operations in accordance with techniques of this disclosure. Examples of processing circuitry 78 may include, any one or more microprocessors, controllers, GPUs, TPUs, DSPs, ASICS, FPGAs, or equivalent discrete or integrated logic circuitry.

Memory 80 may be configured to store information within external device 12, for processing during operation of external device 12. Memory 80, in some examples, is described as a computer-readable storage medium. In some examples, memory 80 includes a temporary memory or a volatile memory. Examples of volatile memories include RAM, DRAM, SRAM, and other forms of volatile memories known in the art. Memory 80, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 80 includes cloud-associated storage.

One or more input devices 82 of external device 12 may receive input, e.g., from a patient, a clinician, or another user. Examples of input are tactile, audio, kinetic, and optical input. Input devices 82 may include, as examples, a mouse, keyboard, voice responsive system, camera, buttons, control pad, microphone, presence-sensitive or touch-sensitive component (e.g., screen), or any other device for detecting input from a user or a machine.

One or more output devices 84 of external device 12 may generate output, e.g., to the patient or another user. Examples of output are tactile, haptic, audio, and visual output. Output devices 84 of external device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 86 may sense physiological parameters or physiological signals of patient 4. Sensor(s) 86 may include electrodes, accelerometers (e.g., 3-axis accelerometers), IMUs, gyroscopes, optical sensors, impedance sensors, temperature sensors, pressure sensors, heart sound sensors (e.g., microphones or accelerometers), and other sensors.

Communication circuitry 88 of external device 12 may communicate with other devices by transmitting and receiving data. Communication circuitry 88 may receive data from IMD 10, such as physiological signals and/or physiological parameter values, from communication circuitry 54 in IMD 10. Communication circuitry 88 may include a network interface card, such as an Ethernet card, an optical transceiver, a radio frequency transceiver, or any other type of device that can send and receive information. For example, communication circuitry 88 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 4, processing circuitry 78 may execute health monitoring application 90 in user space 70 of external device 12. Health monitoring application 90 may be logically divided into presentation layer 92, application layer 94, and data layer 96. Presentation layer 92 may include a user interface (UI) component 98, which generates and renders user interfaces of health monitoring application 90. Data layer 96 may include patient data 100, which may be received from IMD 10 via communication circuitry 88 and stored in memory 80 by processing circuitry 78, or may be determined by processing circuitry 78 and stored in memory 80.

Application layer 94 may include, but is not limited to, a patient condition module 102. In some examples, patient condition module 102 may receive, from IMD 10, an indication of one or more physiological characteristics of patient 4, as determined by IMD 10. Patient condition module 102 may, in response, store the one or more physiological characteristics of patient 4 as patient data 100. In some examples, patient condition module 102 may send, via communication circuitry 88, an indication of the one or more physiological characteristics of patient 4 to a remote system.

In some examples, the one or more physiological characteristics may include whether implanted medical device 13 is operating properly, the motion of implanted medical device 13, whether implanted medical device 13 is or is not deforming as anticipated the rate of blood flow through and/or around implanted medical device 13, the flow type (e.g., laminar flow or turbulent flow) of blood flow through and/or around implanted medical device 13, calcification of a blood vessel, thrombus formation in a blood vessel, blood vessel occlusion, and the like. The physiological characteristics may also include one or more heart conditions of patient 4, such as aortic stenosis or aortic regurgitation, a prediction of heart failure or aneurism, a prediction of when Abdominal Aortic Aneurysm (AAA) grafts or transcatheter aortic valve replacement (TAVR) may be needed for patient 4, and the like.

In some examples, patient condition module 102 may implement any of the techniques described herein to determine one or more physiological characteristics of patient 4. For example, external device 12 may receive, from IMD 10, a signal indicative of sounds and/or vibrations captured by IMD 10. In another example, input device(s) 82 of external device 12 may include a microphone and/or an accelerometer that may monitor for sounds and/or vibrations emitted within the body of patient 4, such as sounds and/or vibrations emitted by implantable medical device 13. Patient condition module 102 may perform any of the techniques performed by processing circuitry 50 of FIG. 4, as described in this disclosure, to determine one or more physiological characteristics of patient 4. Patient condition module 102 may store the one or more physiological characteristics of patient 4 as patient data 100. In some examples, patient condition module 102 may send, via communication circuitry 88, an indication of the one or more physiological characteristics of patient 4 to a remote system. In other examples, patient condition module 102 may store the one or more physiological characteristics of patient 4 as patient data 100. In some examples, patient condition module 102 may send, via communication circuitry 88, the signal indicative of sounds and/or vibrations captured by IMD 10 to a remote system.

In some examples, patient condition module 102 may determine one or more physiological characteristics of patient 4 by using the thermal properties of implantable medical device 13 to evaluate blood flow around implantable medical device 13. For example, there may be differences in thermal properties between turbulent flow and laminar flow and differences in thermal properties for different velocities of laminar flow.

After implantable medical device has been heated, such as via ultrasound or via any other suitable heat delivery system, one or more heat sensors of sensors 86 may be placed proximate to implantable medical device 13 to sense the temperature of implantable medical device 13 over time as implantable medical device 13. For example, sensors 86 may sense the temperature of implantable medical device 13 over time using a thermistor, a thermocouple, a non-contact means via infrared (e.g., FLIR), or by determining impedance changes of implantable medical device 13. External device 12 may be placed over the chest wall of patient 4 to perform transthoracic temperature sensing of implantable medical device.

Processing circuitry 78 may receive, from sensors 86, one or more signals indicative of the temperature of implantable medical device 13 over time, as sensed by sensors 86. Patient condition module 102 may, based on the temperature of implantable medical device 13 over time, monitor the rate of cooling of implantable medical device 13 to determine one or more physiological characteristics of patient 4, such as whether the blood flow around implantable medical device 13 is a laminar flow, the flow velocity of the blood flow, and/or whether the blood flow is turbulent or occluded.

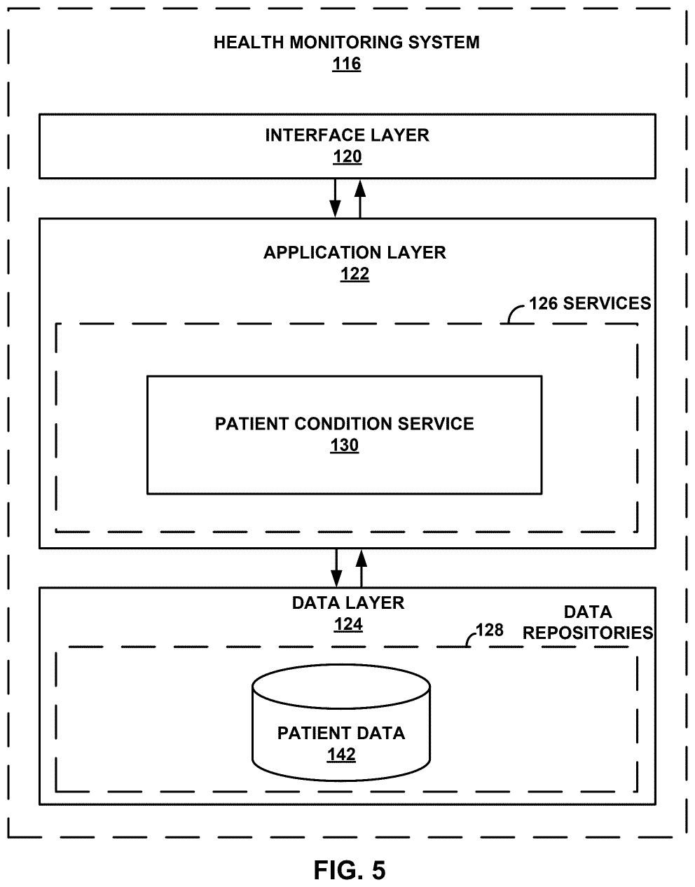

FIG. 5 is a block diagram illustrating an operating perspective of a health monitoring system 116 (“HMS 116”). HMS 116 may be implemented in a computing system 110, which may include hardware components such as processing circuitry 112, memory 114, and communication circuitry, embodied in one or more physical devices. FIG. 5 provides an operating perspective of HMS 116 when hosted as a cloud-based platform. In the example of FIG. 5, components of HMS 116 are arranged according to multiple logical layers that implement the techniques of this disclosure. Each layer may be implemented by one or more modules comprised of hardware, software, or a combination of hardware and software.

Computing devices, such as external device 12, operate as clients that communicate with HMS 116 via interface layer 120. The computing devices typically execute client software applications, such as desktop application(s), mobile application(s), and web application(s). Interface layer 120 represents a set of application programming interfaces (API) or protocol interfaces presented and supported by HMS 116 for the client software applications. Interface layer 120 may be implemented with one or more web servers.

As shown in FIG. 5, HMS 116 also includes an application layer 122 that represents a collection of services 126 for implementing the functionality ascribed to HMS 116 herein. Processing circuitry of HMS 116 may execute application layer 122 receives information from IMD 10 and/or from client applications, e.g., data from external device 12, some or all of which may have been received from IMD 10, and further processes the information according to one or more of services 126. Application layer 122 may be implemented as one or more discrete software services 126 executed on one or more application server, e.g., physical or virtual machines. That is, the application servers provide runtime environments for execution of services 126. In some examples, the functionality of interface layer 120 as described above and the functionality of application layer 122 may be implemented at the same server.

One or more storage devices of HMS 116 may implement data layer 124 of HMS 116. Data layer 124 provides persistence for information in HMS 116 using one or more data repositories 128. A data repository 128, generally, may be any data structure or software that stores and/or manages data. Examples of one or more data repositories 128 include, but are not limited to relational databases, multi-dimensional databases, maps, and/or hash tables.

Software services 126 of application layer 122 includes patient condition service 130. In some examples, patient condition service 130 may receive, from IMD 10 and/or from external device 12, an indication of one or more physiological characteristics of patient 4, as determined by IMD 10 and/or external device 12. Patient condition service 130 may, in response, store the one or more physiological characteristics of patient 4 into one or more data repositories 128 as patient data 142. In some examples, patient condition service 130 may cause HMS 116 to output a notification (e.g., to clinician computing devices, external device 12, and/or to other computing devices and/or systems connected to HMS 116 via a network) that includes an indication of the one or more physiological characteristics of patient 4.

The one or more physiological characteristics may include whether implanted medical device 13 is operating properly, the motion of implanted medical device 13, whether implanted medical device 13 is or is not deforming as anticipated the rate of blood flow through and/or around implanted medical device 13, the flow type (e.g., laminar flow or turbulent flow) of blood flow through and/or around implanted medical device 13, calcification of a blood vessel, thrombus formation in a blood vessel, blood vessel occlusion, and the like. The physiological characteristics may also include one or more heart conditions of patient 4, such as aortic stenosis or aortic regurgitation, a prediction of heart failure or aneurism, a prediction of when Abdominal Aortic Aneurysm (AAA) grafts or transcatheter aortic valve replacement (TAVR) may be needed for patient 4, and the like.

In some examples, patient condition service 130 may implement any of the techniques described herein to determine one or more physiological characteristics of patient 4. For example, HMS 116 may receive, from external device 12 and/or IMD 10, a signal indicative of sounds and/or vibrations captured by IMD 10. Patient condition service 130 may perform any of the techniques performed by processing circuitry 50 of FIG. 4, as described in this disclosure, to determine one or more physiological characteristics of patient 4. Patient condition service 130 may store the one or more physiological characteristics of patient 4 into one or more data repositories 128 as patient data 142. In some examples, patient condition service 130 may cause HMS 116 to output a notification (e.g., to clinician computing devices, external device 12, and/or to other computing devices and/or systems connected to HMS 116 via a network) that includes an indication of the one or more physiological characteristics of patient 4.

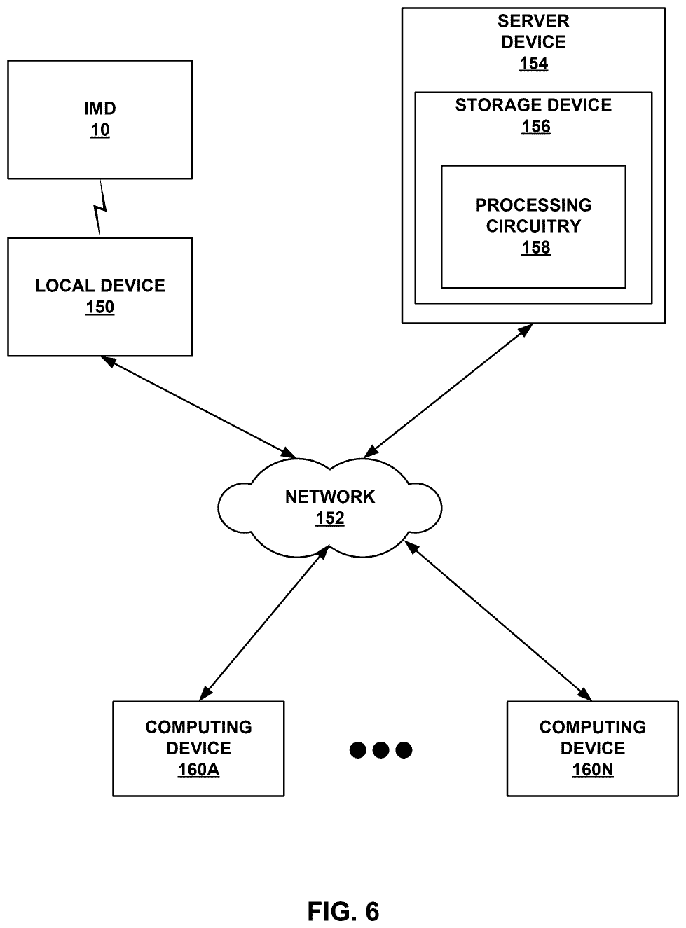

FIG. 6 is a block diagram illustrating an example system that includes a local device 150, a network 152, external computing devices, such as a server 154, and one or more other computing devices 160A-160N (collectively, “computing devices 160”), which may be coupled to IMD 10 and local device 150 via network 152, in accordance with one or more techniques described herein. In this example, IMD 10 may use communication circuitry 54 to communicate with local device 150 via a wireless connection. In the example of FIG. 6, local device 150, external device 12, server 154, and computing devices 160 are interconnected and may communicate with each other through network 152.

Local device 150 may be external device 12, in some examples. Local device 150 may include a device that connects to network 152 via any of a variety of connections, such as telephone dial-up, digital subscriber line (DSL), or cable modem connections. In other examples, local device 150 may be coupled to network 152 through different forms of connections, including wired or wireless connections. In some examples, local device 150 may be a user device, such as a tablet or smartphone, that may be co-located with patient 4. IMD 10 may be configured to transmit data, such as patient data, to local device 150. Local device 150 may then communicate the retrieved data to server 154 via network 152.

In some cases, server 154 may be configured to provide a secure storage site for data that has been collected from IMD 10 and/or external device 12. In some cases, server 154 may assemble data in web pages or other documents for viewing by trained professionals, such as clinicians, via computing devices 160. One or more aspects of the illustrated system of FIG. 6 may be implemented with general network technology and functionality, which may be similar to that provided by the Medtronic CareLink® Network.

In some examples, one or more of computing devices 160 may be a tablet or other smart device located with a clinician, by which the clinician may program, receive alerts from, and/or interrogate IMD 10. For example, the clinician may access patient data and/or indications of patient health collected by IMD 10 through a computing device of computing devices 160, such as when patient 4 is in between clinician visits, to check on a status of a medical condition. In some examples, the clinician may enter instructions for a medical intervention for patient 4 into an application executed by one of computing devices 160, such as based on a status of a patient condition determined by IMD 10, external device 12, server 154, or any combination thereof, or based on other patient data known to the clinician. One of computing devices 160 then may transmit the instructions for medical intervention to another of computing devices 160 located with patient 4 or a caregiver of patient 4. For example, such instructions for medical intervention may include an instruction to change a drug dosage, timing, or selection, to schedule a visit with the clinician, or to seek medical attention. In further examples, one of computing devices 160 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 6, server 154 includes a storage device 156, e.g., to store data retrieved from IMD 10, and processing circuitry 158. Although not illustrated in FIG. 6 computing devices 160 may similarly include a storage device and processing circuitry.

Storage device 156 may include a computer-readable storage medium or computer-readable storage device. In some examples, storage device 156 includes one or more of a short-term memory or a long-term memory. Storage device 156 may include, for example, RAM, DRAM, SRAM, magnetic discs, optical discs, flash memories, or forms of EPROM or EEPROM. In some examples, storage device 156 is used to store data indicative of instructions for execution by processing circuitry 158.

Processing circuitry 158 may include one or more processors that are configured to implement functionality and/or process instructions for execution within server 154. For example, processing circuitry 158 may be capable of processing instructions stored in storage device 156. Processing circuitry 158 may include, for example, microprocessors, DSPs, ASICS, FPGAs, or equivalent discrete or integrated logic circuitry, or a combination of any of the foregoing devices or circuitry. Accordingly, processing circuitry 158 may include any suitable structure, whether in hardware, software, firmware, or any combination thereof, to perform the functions ascribed herein to processing circuitry 158.

Processing circuitry 158 of server 154 and/or the processing circuitry of computing devices 160 may implement any of the techniques described herein to determine one or more physiological characteristics of patient 4. For example, server 154 and/or any one of computing devices 160 may receive, from external device 12, a signal indicative of sounds and/or vibrations captured by IMD 10. Processing circuitry 158 and/or the processing circuitry of computing devices 160 may perform any of the techniques performed by processing circuitry 50 of FIG. 4, as described in this disclosure, to determine one or more physiological characteristics of patient 4.

Processing circuitry 158 of server 154 and/or the processing circuitry of computing devices 160 may output an indication of the one or more physiological characteristics of patient 4. For example, server 154 may transmit, to one or more of computing devices 160, a signal indicative of one or more physiological characteristics of patient 4. In another example, one of computing devices 160 may transmit, to another one of computing devices 160, a signal indicative of one or more physiological characteristics of patient 4.

In some examples, one of computing devices 160 may output a graphical user interface that includes information regarding the one or more physiological characteristics of patient 4. The one or more physiological characteristics may include whether implanted medical device 13 is operating properly, the motion of implanted medical device 13, whether implanted medical device 13 is or is not deforming as anticipated the rate of blood flow through and/or around implanted medical device 13, the flow type (e.g., laminar flow or turbulent flow) of blood flow through and/or around implanted medical device 13, calcification of a blood vessel, thrombus formation in a blood vessel, blood vessel occlusion, and the like. The physiological characteristics may also include one or more heart conditions of patient 4, such as aortic stenosis or aortic regurgitation, a prediction of heart failure or aneurism, a prediction of when Abdominal Aortic Aneurysm (AAA) grafts or transcatheter aortic valve replacement (TAVR) may be needed for patient 4, and the like.

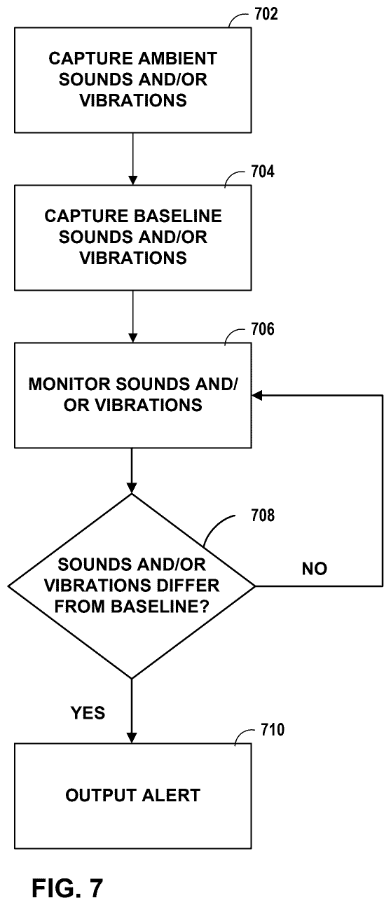

FIG. 7 is a flow diagram illustrating an example technique of determining a physiological characteristic of the patent. FIG. 7 is described with respect to IMD 10.

As shown in FIG. 7, after IMD is implanted in the body of patient 4 but before implanted medical device 13 is implanted in the body of patient 4, sensors 62 of IMD 10 may capture ambient sounds and/or vibrations emitted within the heart of patient 4 (702). After implanted medical device 13 is implanted in the body of patient 4, accelerometer 65 and/or microphone 67 of IMD 10 may capture sounds and/or vibrations that are emitted within the body of patient 4 (704).

Such sounds and/or vibrations that are emitted within the body of patient 4 after implantation of implanted medical device 13 is referred to as a baseline signal for implanted medical device 13. Processing circuitry 50 may determine, based on sounds and/or vibrations that are emitted within the body of patient 4 after implantation of implanted medical device 13, a corresponding signature for the baseline signal for implanted medical device 13. As described above, a signature for the baseline signal for implanted medical device 13 may be a specific pattern of sounds and/or vibrations, which may include parameters such as frequency, amplitude, phase, duration, intensity, timing, rhythm, and the like.

After determining the baseline signal for implanted medical device 13, IMD 10 may use accelerometer 65 and/or microphone 67 to continuously monitor and capture sounds and/or vibrations that are emitted within the heart of patient 4, and sensors 62 may generate signals indicative of the sounds and/or vibrations captured by accelerometer 65 and/or microphone 67 (706).

Processing circuitry 50 of IMD 10 may obtain the signals generated by sensors 62 indicative of the sounds and/or vibrations captured by accelerometer 65 and/or microphone 67 and may compare the captured sounds and/or vibrations against the baseline signal to determine whether the captured sounds and/or vibrations differ from the baseline sound and/or baseline vibration (708). For example, processing circuitry 50 may compare one or more characteristics of the sounds and/or vibrations captured by accelerometer 65 and/or microphone 67, such as a pattern of the captured sounds and/or vibrations, which may include parameters such as frequency, amplitude, phase, duration, intensity, timing, rhythm, and the like, against the signature for the baseline signal for implanted medical device 13 to determine one or more differences between the sounds and/or vibrations captured by accelerometer 65 and/or microphone 67 and the baseline signal. Such differences may be indicative of one or more physiological characteristics of patient 4.

Processing circuitry 50 may, in response to determining that the captured sounds and/or vibrations differ from the baseline sound and/or baseline vibration, output an alert indicative of the difference between the captured sounds and/or vibrations and the baseline sound and/or baseline vibration, such as to external device 12 or to a remote server (710). For example, the alert may indicate one or more physiological characteristics of patient 4, such as whether implanted medical device 13 is operating properly, the motion of implanted medical device 13, whether implanted medical device 13 is or is not deforming as anticipated the rate of blood flow through and/or around implanted medical device 13, the flow type (e.g., laminar flow or turbulent flow) of blood flow through and/or around implanted medical device 13, calcification of a blood vessel, thrombus formation in a blood vessel, blood vessel occlusion, and the like.

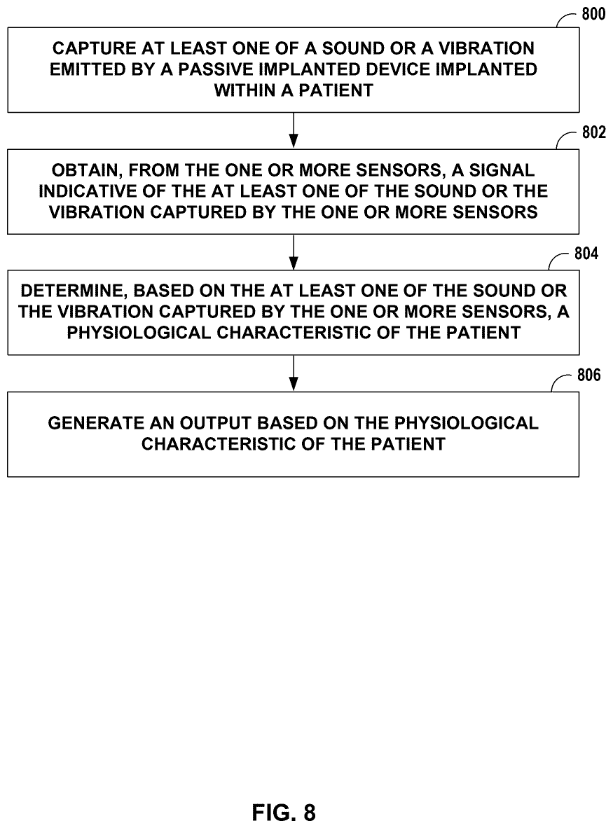

FIG. 8 is a flow diagram that illustrates performing an example technique according to this disclosure. Although primarily described with respect to IMD 10, it should be understood that the techniques of this disclosure may be applied to any medical device described herein.

As shown in FIG. 8, one or more sensors 62 of IMD 10 may capture at least one of a sound or a vibration emitted by a passive implanted device 13 implanted within a patient 4 (802). In some examples, the one or more sensors 62 include one or more of an accelerometer 65 or an audio sensor 67. Processing circuitry 50 of IMD 10 may obtain, from the one or more sensors 62, a signal indicative of the at least one of the sound or the vibration captured by the one or more sensors 62 (804).

Processing circuitry 50 may determine, based on the at least one of the sound or the vibration captured by the one or more sensors 62, a physiological characteristic of the patient 4 (806). In some examples, to determine the physiological characteristic of the patient 4, the processing circuitry 50 may filter the at least one of the sound or the vibration captured by the one or more sensors 62 to extract a specific range of frequencies from the sound or the vibration captured by the one or more sensors 62. Processing circuitry 50 may apply a high-pass filter to the at least one of the sound or the vibration captured by the one or more sensors 62 to extract frequencies above a cutoff frequency from the sound or the vibration captured by the one or more sensors 62. In some examples, the cutoff frequency is 100 Hertz. In some examples, the cutoff frequency is 1000 Hertz.

In some examples, to determine the physiological characteristic of the patient, the processing circuitry 50 may determine a specific pattern of sounds or vibrations from the at least one of the sound or the vibration captured by the one or more sensors 62 and may compare the specific pattern of sounds or vibrations from the at least one of the sound or the vibration captured by the one or more sensors 62 with signatures corresponding to a plurality of physiological characteristics to determine the physiological characteristic of the patient 4.

Processing circuitry 50 may generate an output based on the physiological characteristic of the heart of the patient 4 (808).

In some examples, the passive implanted device 13 is implanted in the heart of the patient 4. In some examples, the passive implanted device 13 is a stent, a prosthetic valve, a graft, or an atrial appendage occlusion device.

In some examples, the at least one of the sound or the vibration emitted by the passive implanted device 13 is indicative of physical deformation of the passive implanted device 13. In some examples, the at least one of the sound or the vibration is emitted by a speaker device of the passive implanted device 13 that is powered by an piezoelectric element. In some examples, the one or more physiological characteristics of the patient 4 comprise one or more of a flow rate of blood through a vessel or a flow type of the blood through the vessel.

In some examples, the one or more sensors 62 may capture at least one of a baseline sound or a baseline vibration emitted by the passive implanted device 13. To determine the physiological characteristic of the heart of the patient 4, the processing circuitry 50 may compare the at least one of the baseline sound or the baseline vibration emitted by the passive implanted device 13 with the at least one of the sound or the vibration emitted by the passive implanted device 13 to determine the physiological characteristic of the heart of the patient 4.

In some examples, the implantable medical device 10 is an insertable cardiac monitor. In some examples, the insertable cardiac monitor 10 includes a housing configured for subcutaneous implantation in the patient 4, the housing having a length between 40 millimeters (mm) and 60 mm between a first end and a second end, a width less than the length, and a depth less than the width.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

What is claimed is:

1. An implantable medical device comprising:
one or more sensors configured to capture at least one of a sound or a vibration emitted by a passive implanted device implanted within a patient; and
processing circuitry configured to:
obtain, from the one or more sensors, a signal indicative of the at least one of the sound or the vibration captured by the one or more sensors;
determine, based on the at least one of the sound or the vibration captured by the one or more sensors, a physiological characteristic of the patient; and
generate an output based on the physiological characteristic of the patient.

2. The implantable medical device of claim 1, wherein the one or more sensors include one or more of an accelerometer or an audio sensor.

3. The implantable medical device of claim 1, wherein to determine the physiological characteristic of the patient, the processing circuitry is further configured to:
filter the at least one of the sound or the vibration captured by the one or more sensors to extract a specific range of frequencies from the sound or the vibration captured by the one or more sensors.

4. The implantable medical device of claim 3, wherein to filter the at least one of the sound or the vibration captured by the one or more sensors to extract the specific range of frequencies from the sound or the vibration captured by the one or more sensors, the processing circuitry is further configured to:
apply a high-pass filter to the at least one of the sound or the vibration captured by the one or more sensors to extract frequencies above a cutoff frequency from the sound or the vibration captured by the one or more sensors.

5. The implantable medical device of claim 1, wherein to determine the physiological characteristic of the patient, the processing circuitry is further configured to:
determine a specific pattern of sounds or vibrations from the at least one of the sound or the vibration captured by the one or more sensors; and
compare the specific pattern of sounds or vibrations from the at least one of the sound or the vibration captured by the one or more sensors with signatures corresponding to a plurality of physiological characteristics to determine the physiological characteristic of the patient.

6. The implantable device of claim 1, wherein the passive implanted device is implanted in the heart of the patient.

7. The implantable device of claim 1, wherein the at least one of the sound or the vibration emitted by the passive implanted device is indicative of physical deformation of the passive implanted device.

8. The implantable device of claim 1, wherein the at least one of the sound or the vibration is emitted by a speaker device of the passive implanted device that is powered by an piezoelectric element.

9. The implantable medical device of claim 1, wherein the physiological characteristic of the patient comprise one or more of a flow rate of blood through a vessel or a flow type of the blood through the vessel.

10. The implantable medical device of claim 1, wherein the one or more sensors are further configured to capture at least one of a baseline sound or a baseline vibration emitted by the passive implanted device, and wherein to determine the physiological characteristic of the patient, the processing circuitry is further configured to:
compare the at least one of the baseline sound or the baseline vibration emitted by the passive implanted device with the at least one of the sound or the vibration emitted by the passive implanted device to determine the physiological characteristic of the patient.

11. The implantable medical device of claim 1, wherein the passive implanted device comprises a stent, a prosthetic valve, a graft, or an atrial appendage occlusion device.

12. The implantable medical device of claim 1, wherein the implantable medical device comprises an insertable cardiac monitor.

13. The implantable medical device of claim 12, wherein the insertable cardiac monitor comprises:
a housing configured for subcutaneous implantation in the patient, the housing having a length between 40 millimeters (mm) and 60 mm between a first end and a second end, a width less than the length, and a depth less than the width.

14. A method comprising:
capturing, by one or more sensors of an implantable medical device, at least one of a sound or a vibration emitted by a passive implanted device implanted within a patient;
obtaining, by processing circuitry and from the one or more sensors, a signal indicative of the at least one of the sound or the vibration captured by the one or more sensors;
determining, by the processing circuitry and based on the at least one of the sound or the vibration captured by the one or more sensors, a physiological characteristic of the patient; and
generating, by the processing circuitry, an output based on the physiological characteristic of the patient.

15. The method of claim 14, wherein the one or more sensors include one or more of an accelerometer or an audio sensor.

16. The method of claim 14, wherein determining the physiological characteristic of the patient further comprises:
filtering, by the processing circuitry, the at least one of the sound or the vibration captured by the one or more sensors to extract a specific range of frequencies from the sound or the vibration captured by the one or more sensors.

17. The method of claim 16, wherein filtering the at least one of the sound or the vibration captured by the one or more sensors to extract the specific range of frequencies from the sound or the vibration captured by the one or more sensors further comprises:
applying, by the processing circuitry, a high-pass filter to the at least one of the sound or the vibration captured by the one or more sensors to extract frequencies above a cutoff frequency from the sound or the vibration captured by the one or more sensors.

18. The method of claim 17, wherein determining the physiological characteristic of the patient further comprises:
determining a specific pattern of sounds or vibrations from the at least one of the sound or the vibration captured by the one or more sensors; and
comparing the specific pattern of sounds or vibrations from the at least one of the sound or the vibration captured by the one or more sensors with signatures corresponding to a plurality of physiological characteristics to determine the physiological characteristic of the patient.

19. The method of claim 14, further comprising:
capturing, by the one or more sensors, at least one of a baseline sound or a baseline vibration emitted by the passive implanted device;
wherein determining the physiological characteristic of the patient further comprises comparing, by the processing circuitry, the at least one of the baseline sound or the baseline vibration emitted by the passive implanted device with the at least one of the sound or the vibration emitted by the passive implanted device to determine the physiological characteristic of the patient.

20. A system comprising:
an implantable medical device comprising one or more sensors configured to capture at least one of a sound or a vibration emitted by a passive implanted device implanted within a patient; and
an external device comprising processing circuitry configured to:
obtain, from the one or more sensors, a signal indicative of the at least one of the sound or the vibration captured by the one or more sensors;
determine, based on the at least one of the sound or the vibration captured by the one or more sensors, a physiological characteristic of the patient; and
generate an output based on the physiological characteristic of the patient.
